(19) United States
(12) Patent Application Publication
(10) Pub. No.: US 2022/0022817 A1
(43) Pub. Date: Jan. 27, 2022

(54) CARDIAC PHASE PREDICTION IN CARDIAC MRI USING DEEP LEARNING

(71) Applicant: Shanghai United Imaging Intelligence Co., LTD., Shanghai (CN)

(72) Inventors: Zhang Chen, Cambridge, MA (US); Shanhui Sun, Cambridge, MA (US); Xiao Chen, Cambridge, MA (US); Terrence Chen, Cambridge, MA (US)

(73) Assignee: Shanghai United Imaging Intelligence Co., LTD., Shanghai (CN)

(21) Appl. No.: 16/934,623

(22) Filed: Jul. 21, 2020

(51) Int. Cl. A61B 5/00; A61B 5/055; A61B 5/0452; G01R 33/48

(52) U.S. Cl. CPC A61B 5/7275; A61B 5/055; G01R 33/4818; A61B 5/0452; A61B 5/7264

(57) ABSTRACT

A method includes acquiring MRI data, using an algorithm to predict cardiac cycles from the acquired MRI data, and operating on sections of the acquired MRI data corresponding to selected portions of the predicted cardiac cycles.

### BACKGROUND

The aspects of the present disclosure relate generally to Magnetic resonance imaging (MRI), and in particular to predicting cardiac signals from MRI data.

MRI is a widely used medical technique which produces images of a region of interest using magnetic and radio frequency energy. During an MRI scan, volume coils (for example, body coils) and local coils (for example, surface coils) may acquire MR signals produced by nuclear relaxation inside the object being examined. Cardiac MRI may be used to produce detailed pictures of the structures within and around the heart for evaluating the heart's anatomy and function and for detecting or monitoring cardiac disease. The output of a cardiac MRI procedure may be MRI data in the form of k-space data converted to cardiac cine. In many applications of cardiac MRI, electrical signals produced by the heart are acquired separately and used as reference points for identifying cardiac phase information or timing in the cardiac cine.

For example, in cardiac MRI image reconstruction, the cardiac phase information or timing may be used to trim the MRI image to a single cardiac cycle. FIG. 1 shows a typical work flow 100 where MRI data is acquired 102 and cardiac cine are reconstructed from the MRI data 104. A separately acquired ECG signal 106 is acquired and manually correlated with the images 104 in order to determine whether to remove or keep certain cardiac phases of the MRI data 108. In other post-processing applications, such as cardiac strain analysis, the shape and function of the heart muscles may be of interest during end diastolic and/or end systolic phases of the cardiac cycle. FIG. 2 illustrates a typical work flow 200 where MRI data in the form of previously acquired MRI images 202 may be manually labelled to identify cardiac phases, for example, end diastolic and/or end systolic phases 204, and then used for analysis 206, for example, cardiac strain analysis.

However, correlation between the cardiac signals and the MRI data generally requires conversion of the MRI data to cardiac cine and then requires the expertise of a trained medical professional to manually correlate the MRI detected cardiac activity with a particular type of cardiac signal. These manual activities may be time and labor intensive and may be subject to inconsistencies.

### SUMMARY

It would be advantageous to provide a method and system that automatically correlates MRI data and cardiac signals consistently and without manual intervention.

According to an aspect of the present disclosure, a method includes acquiring MRI data, using an algorithm to predict cardiac cycles from the acquired MRI data, and operating on sections of the acquired MRI data corresponding to selected portions of the predicted cardiac cycles.

The acquired MRI data may include k space data.

The acquired MRI data may include image data.

The acquired MRI data may include under sampled MRI data.

The acquired MRI data may include ECG signals from a subject under study captured during MRI scanning.

The acquired MRI data may include video images of a subject under study captured during MRI scanning.

The acquired MRI data may include pulse data from a subject under study captured during MRI scanning.

The algorithm may include a deep learning model further including one or more of a combination CNN and RNN models, a GRU model, an LSTM model, a fully convolutional neural network model, a generative adversarial network, a back propagation neural network model, a radial basis function neural network model, a deep belief nets neural network model, an Elman neural network model.

Operating on sections of the acquired MRI data corresponding to selected portions of the predicted cardiac cycle may include positioning data lines in a k-space of the acquired MRI data.

Operating on sections of the acquired MRI data corresponding to selected portions of the predicted cardiac cycle may include interpolating between MRI data lines in a k-space of the acquired MRI data.

Operating on sections of the acquired MRI data corresponding to selected portions of the predicted cardiac cycle may include interpolating between MRI images.

Operating on sections of the acquired MRI data corresponding to selected portions of the predicted cardiac cycle may include performing cardiac strain analysis using the sections of the acquired MRI data.

Operating on sections of the acquired MRI data corresponding to selected portions of the predicted cardiac cycle may include performing cine image reconstruction on the sections of the acquired MRI data.

The method may further include acquiring a cardiac signal corresponding to the MRI data, and using the algorithm to predict the one or more predicted cardiac signals from the MRI acquired data and the acquired cardiac signal.

The predicted portions of cardiac cycles may represent any portions of the cardiac cycles.

The predicted portions of cardiac cycles may represent one or more of end systole or end diastole cardiac phases.

The predicted portions of cardiac cycles may represent one or more of one or more of P, Q, R, S, T, U, QRS complex, or PR interval cardiac waves.

According to an aspect of the present disclosure a system includes receive and control circuitry operating an algorithm configured to predict cardiac cycles from MRI data, and a processing engine configured to operate on sections of the MRI data corresponding to selected portions of the predicted cardiac cycles.

These and other aspects, implementation forms, and advantages of the exemplary embodiments will become apparent from the embodiments described herein considered in conjunction with the accompanying drawings. It is to be understood, however, that the description and drawings are designed solely for purposes of illustration and not as a definition of the limits of the disclosed invention, for which reference should be made to the appended claims. Additional aspects and advantages of the invention will be set forth in the description that follows, and in part will be obvious from the description, or may be learned by practice of the invention. Moreover, the aspects and advantages of the invention may be realized and obtained by means of the instrumentalities and combinations particularly pointed out in the appended claims.

### BRIEF DESCRIPTION OF THE DRAWINGS

In the following detailed portion of the present disclosure, the invention will be explained in more detail with reference to the example embodiments shown in the drawings. These embodiments are non-limiting exemplary embodiments, in which like reference numerals represent similar structures throughout the several views of the drawings, wherein:

FIG. 1 illustrates a typical work flow where MRI data is acquired and cardiac cine are reconstructed from the MRI data;

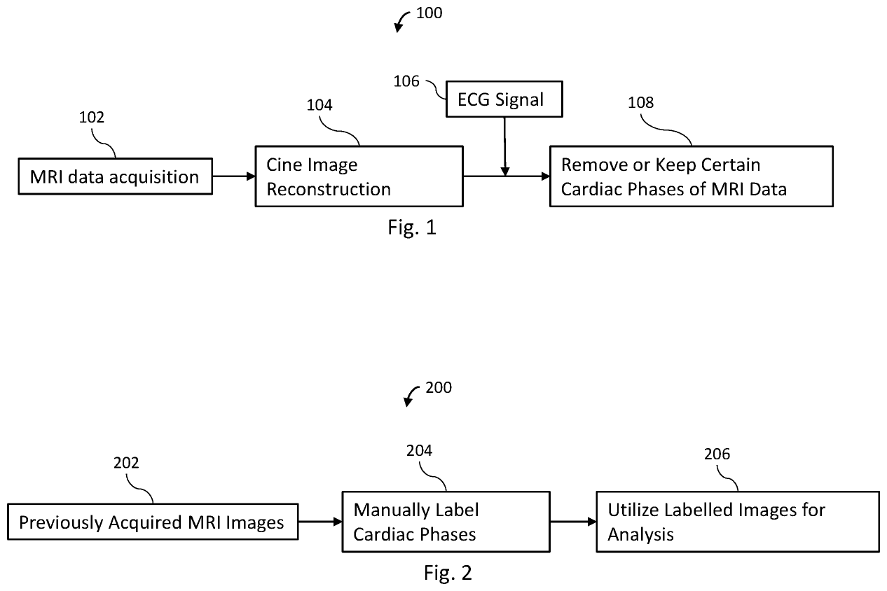

FIG. 2 illustrates a typical work flow where MRI data in the form of previously acquired MRI images may be manually labelled to identify cardiac phases and then used for analysis;

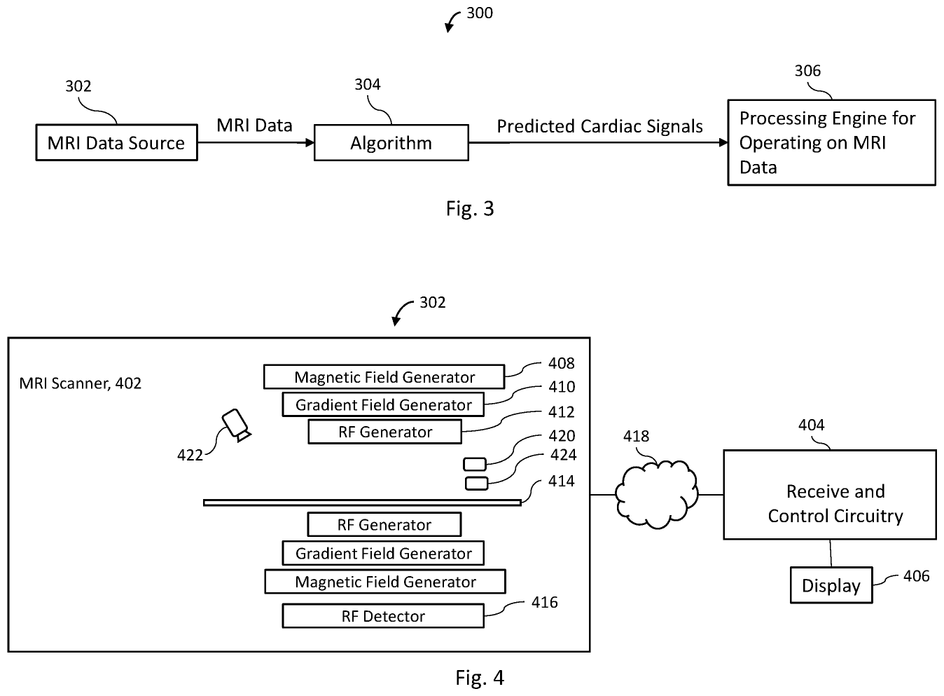

FIG. 3 illustrates an exemplary process flow according to aspects of the disclosed embodiments;

FIG. 4 illustrates an exemplary MRI apparatus according to aspects of the disclosed embodiments;

FIG. 5 shows exemplary MRI data sources for implementing the disclosed embodiments;

FIG. 6 illustrates an exemplary deep learning model according to aspects of the disclosed embodiments;

FIG. 7 illustrates an exemplary architecture of the processing engine 306 according to the disclosed embodiments; and

8-13 illustrate exemplary process flows according to aspects of the disclosed embodiments.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirits and scope of the present disclosure. Thus, the present disclosure is not limited to the embodiments shown, but to be accorded the widest scope consistent with the claims.

It will be understood that the term “system,” “unit,” “module,” and/or “block” used herein are one method to distinguish different components, elements, parts, section or assembly of different level in ascending order. However, the terms may be displaced by other expression if they may achieve the same purpose.

It will be understood that when a unit, module or block is referred to as being “on,” “connected to” or “coupled to” another unit, module, or block, it may be directly on, connected or coupled to the other unit, module, or block, or intervening unit, module, or block may be present, unless the context clearly indicates otherwise. As used herein, the term “and/or” includes any and all combinations of one or more of the associated listed items.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an Erasable Programmable Read Only Memory (EPROM). It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

The terminology used herein is for the purposes of describing particular examples and embodiments only, and is not intended to be limiting. As used herein, the singular forms “a,” “an,” and “the” may be intended to include the plural forms as well, unless the context clearly indicates otherwise. It will be further understood that the terms “include,” and/or “comprise,” when used in this disclosure, specify the presence of integers, devices, behaviors, stated features, steps, elements, operations, and/or components, but do not exclude the presence or addition of one or more other integers, devices, behaviors, features, steps, elements, operations, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The disclosed embodiments are directed to a method comprising acquiring MRI data, using an algorithm to predict cardiac cycles from the acquired MRI data, and operating on sections of the acquired MRI data corresponding to selected portions of the predicted cardiac cycles.

The disclosed embodiments are further directed to a system comprising receiving and control circuitry operating an algorithm configured to predict cardiac cycles from MRI data, and a processing engine configured to operate on sections of the MRI data corresponding to selected portions of the predicted cardiac cycles.

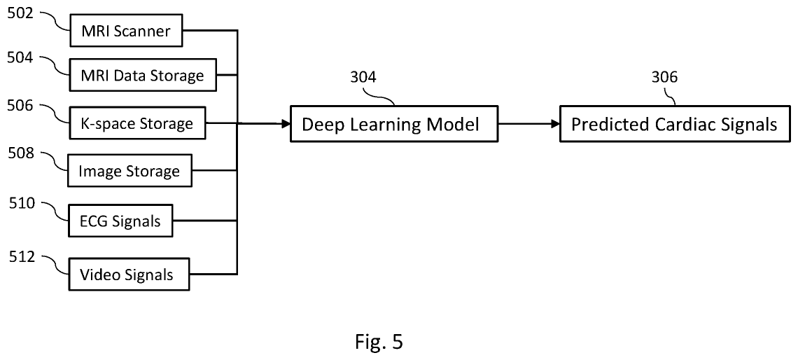

Referring to FIG. 3, a schematic block diagram of an exemplary system 300 incorporating aspects of the disclosed embodiments is illustrated. The system may include an MRI data source 302 for providing MRI data, for example, one or more of cardiac k-space data, cardiac image data, a cardiac time series if images, ECG data, or other recurring data. An algorithm 304 may produce predicted portions of cardiac signals from the MRI data, and a processing engine 306 may operate on portions of the acquired MRI data corresponding to selected ones of the predicted cardiac cycle portions. It should be understood that the components of the system 300 may be implemented in hardware, software, or a combination of hardware and software.

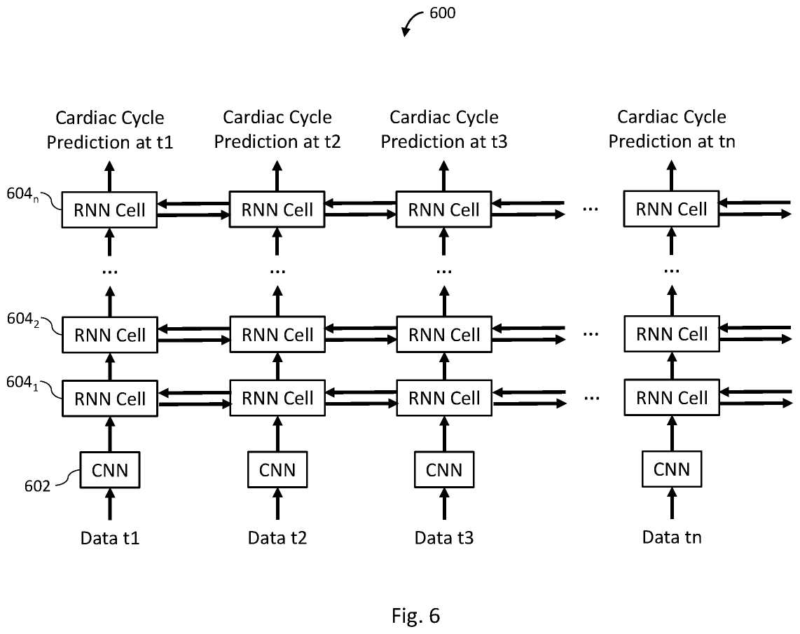

FIG. 4 shows a schematic block diagram of an exemplary MRI apparatus 302 for providing MRI data according to the disclosed embodiments. The MRI apparatus 302 may include an MRI scanner 402, control circuitry 404 and a display 406. The MRI scanner 402 may include, as shown in cross section in FIG. 4, a magnetic field generator 408, a gradient magnetic field generator 410, and a Radio Frequency (RF) generator 412, all surrounding a table 414 on which subjects under study may be positioned. The MRI scanner 402 may also include an ECG signal sensor 420 for capturing MRI data in the form of ECG signals from the subject under study during MRI scanning, a camera 422 for capturing MRI data in the form of video images of the subject under study during MRI scanning, and a pulse detector 424, for capturing MRI data in the form of a subject's pulse during MRI scanning. In some embodiments, the MRI scanner 402 may perform a scan on a subject or a region of the subject. The subject may be, for example, a human body or other animal body. For example, the subject may be a patient. The region of the subject may include part of the subject. For example, the region of the subject may include a tissue of the patient. The tissue may include, for example, lung, prostate, breast, colon, rectum, bladder, ovary, skin, liver, spine, bone, pancreas, cervix, lymph, thyroid, spleen, adrenal gland, salivary gland, sebaceous gland, testis, thymus gland, penis, uterus, trachea, skeletal muscle, smooth muscle, heart, etc. In some embodiments, the scan may be a pre-scan for calibrating an imaging scan. In some embodiments, the scan may be an imaging scan for generating an image.

The main magnetic field generator 408 may create a static magnetic field Bo and may include, for example, a permanent magnet, a superconducting electromagnet, a resistive electromagnet, or any magnetic field generation device suitable for generating a static magnetic field. The gradient magnet field generator 410 may use coils to generate a magnetic field in the same direction as Bo but with a gradient in one or more directions, for example, along X, Y, or Z axes in a coordinate system of the MRI scanner 402.

In some embodiments, the RF generator 412 may use RF coils to transmit RF energy through the subject, or region of interest of the subject, to induce electrical signals in the region of interest. The resulting RF field is typically referred to as the Bi field and combines with the Bo field to generate MR signals that are spatially localized and encoded by the gradient magnetic field. The MRI scanner 402 may further include an RF detector 416 implemented using, for example, an RF coil, where the RF detector operates to sense the RF field and convey a corresponding output to the receive and control circuitry 404. The function, size, type, geometry, position, amount, or magnitude of the MRI scanner 402 may be determined or changed according to one or more specific conditions. For example, the MRI scanner 402 may be designed to surround a subject (or a region of the subject) to form a tunnel type MRI scanner, referred to as a closed bore MRI scanner, or an open MRI scanner, referred to as an open-bore MRI scanner.

The ECG signal sensor 420 may operate to capture ECG signals from the subject under study during MRI scanning for use by the algorithm 304 in subsequently identifying cardiac cycles and cardiac phases of the subject. The camera 422 may operate to capture video images of the subject under study during MRI scanning for use by the algorithm 304 in subsequently identifying cardiac cycles and cardiac phases of the subject. During MRI scanning the subject may be requested to hold their breath and to stay still in order to provide accurate MRI cardiac data while scanning. However, this may be difficult for any number of reasons, and video images of the subject may be used as an input to the algorithm 304 to further enhance cardiac cycle and phase predictions, in particular to compensate for subject movement or breathing patterns during scanning that may adversely affect the acquired MRI data. The pulse detector 424 may provide pulse data from the subject during MRI scanning which may also be used as an input to the algorithm 304 to further enhance cardiac cycle and phase predictions.

The receive and control circuitry 404 may control overall operations of the MRI scanner 402, in particular, the magnetic field generator 408, the gradient magnetic field generator 410, the RF generator 412, and the RF detector 416. For example, the receive and control circuitry 404 may control the magnet field gradient generator to produce gradient fields along one or more of the X, Y, and Z axes, and the RF generator to generate the RF field. In some embodiments, the receive and control circuitry 404 may receive commands from, for example, a user or another system, and control the magnetic field generator 408, the gradient magnetic field generator 410, the RF generator 412, and the RF detector 416 accordingly. The receive and control circuitry 404 may be connected to the MRI scanner 402 through a network 418. The network 418 may include any suitable network that can facilitate the exchange of information and/or data for the MRI scanner 402. The network 418 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 418 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 418 may include one or more network access points. For example, the network 418 may include wired and/or wireless network access points such as base stations and/or internet exchange points through which one or more components of the MRI scanner 402 may be connected with the network 418 to exchange data and/or information.

According to some embodiments, the receive and control circuitry 404 may operate the algorithm 304 for predicting one or more cardiac signals from the MRI acquired data and may include the processing engine 306 for operating on the MRI data. According to one or more embodiments, the receive and control circuitry 404 and the processing engine 306 may be located remotely from the receive and control circuitry.

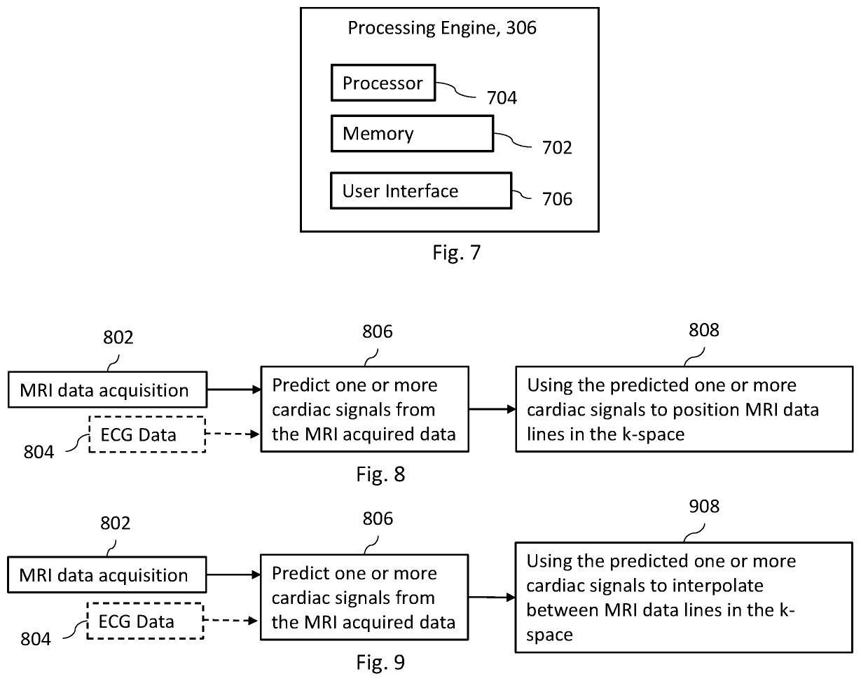

FIG. 5 shows exemplary MRI data sources for implementing the disclosed embodiments. The sources of MRI data may include, without limitation, one or more of an MRI scanner 502, a storage of MRI data 504, for example, MRI slices or other MRI apparatus output, storage of k-space data 506 from any number of MRI scans, an image storage 508, a storage of ECG signals 510, a storage of video images of the subject under study captured during MRI scanning, or any other source of MRI data. In some embodiments, the MRI data may include any type of MRI images, any type of recurring sequential data, for example, video data with individual images along a series of time points, k-space data with k-spaces along a series of time points, sequential ECG data over a series of time points, a combination of one or more of the video data, k-space data, and sequential ECG data, undersampled MRI data, and any other sequential or recurring data from which portions of cardiac cycles may be predicted. In at least one embodiment, the data in the image storage 508 may include Digital Imaging and Communication in Medicine (DICOM®) images. The MRI data sources may further include any number of local, remote, or cloud based sources.

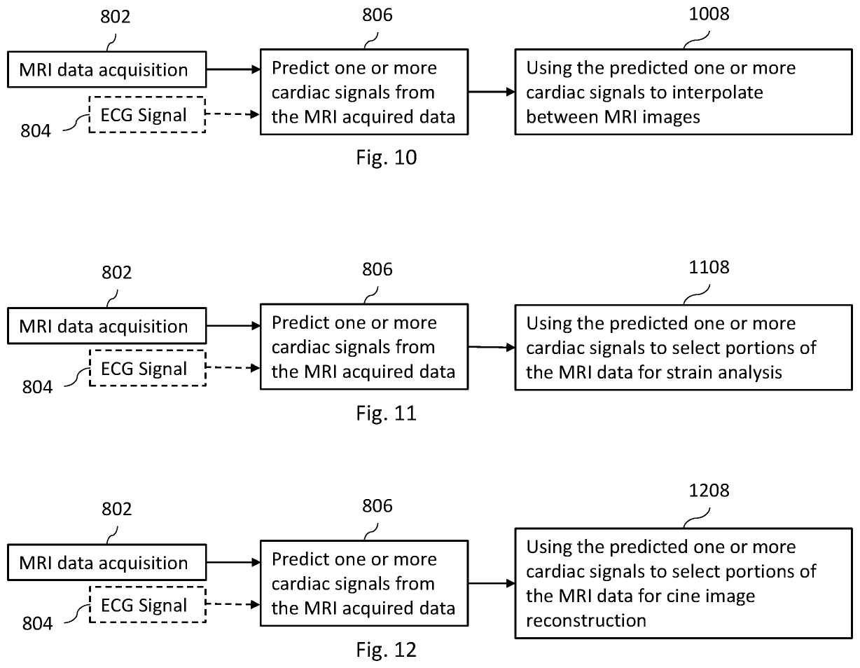

FIG. 6 illustrates an example of the algorithm 304 utilized in the form of a deep learning model 600. The deep learning model 600 generally operates to predict portions of cardiac cycles signals directly from the MRI data. In this example, the deep learning model 600 may include a number of convolutional neural networks 602 and recurrent neural network layers 604₁-604ₙ. MRI data points Data t1, Data t2, . . . Data tn, from one or more of the MRI data sources 302 are provided to the convolutional neural networks 602. As mentioned above, the MRI data points Data t1, Data t2, . . . Data tn may include video data with individual images along each of a series of time points t1, t2, . . . tn, k-space data with k-spaces along a series of time points, DICOM images, sequential ECG data over a series of time points, and any other sequential or recurring data from which portions of cardiac cycles may be predicted, alone or in any combination. The convolutional neural networks 602 operate to pre-process the MRI data and provide the pre-processed sequential data to the recurrent neural network layers 604₁-604ₙ. The multiple recurrent neural network layers 604₁-604ₙ may process the pre-processed sequential data to yield a prediction of a portion of a cardiac cycle for each time point t1, t2, . . . tn. In some embodiments, the convolutional neural networks 602 may operate to extract features from the images or k-space data along a series of time points and the recurrent neural network layers 604₁-604ₙ may utilize the extracted features to predict the cardiac phases or ECG signals. The predicted portions of cardiac cycles may represent any portions of the cardiac cycles. The predicted portions of cardiac cycles may also represent one or more of end systole or end diastole cardiac phases. Furthermore, the predicted portions of cardiac cycles may represent one or more of one or more of P, Q, R, S, T, U, QRS complex, or PR interval cardiac phases.

The deep learning model 600 may operate to predict cardiac cycles in a prospective fashion or may operate to predict cardiac cycles in a retrospective fashion.

When predicting in a prospective fashion, the current cardiac phase of a whole cardiac cycle is predicted using the data collected at and before the current time point. During model training, the model is fed with input data (MRI data, ECG data, pulse data etc.) and ground truth labels in the form of annotated phases of the cardiac cycles. No data after the current time point is provided. The model is trained to learn the relationship between the input data and labels. During inference time, the trained model is used to predict the current phase in the cardiac cycles based on the data that have been collected so far.

When predicting in a retrospective fashion, the cardiac phases in the middle of the cardiac cycles are predicted using the input data after all the data have been collected, for example, during postprocessing steps. That is, the model can use the data collected after the predicted phase. The training and inference procedure are similar to prospective cases, except the data can be collected after the predicted phase.

Regarding the cardiac cycle prediction: the heart repeats the pattern between diastole and systole stages. These two stages are often considered as landmarks to divide the cardiac cycle into phases. It should be understood that “predicting the cardiac cycle” refers to the model having the capability to predict any cardiac stage/phase, not limited to diastole and systole phases. The prediction can be in any format. For example, the model may be used to predict if the current data represents a diastole phase. It is also possible to utilize a continuing number series (for example, 0, 0.1, 0.2 etc.) in the prediction of the cardiac cycles or phases.

While the deep learning model 600 is shown and described as including a combination of convolutional neural networks and recurrent neural networks, it should be understood that the deep learning model may include one or more gated recurrent units (GRUs), long short term memory (LSTM) networks, fully convolutional neural network (FCN) models, generative adversarial networks (GANs), back propagation (BP) neural network models, radial basis function (RBF) neural network models, deep belief nets (DBN) neural network models, Elman neural network models, or any deep learning or machine learning model capable of performing the operations described herein may be used.

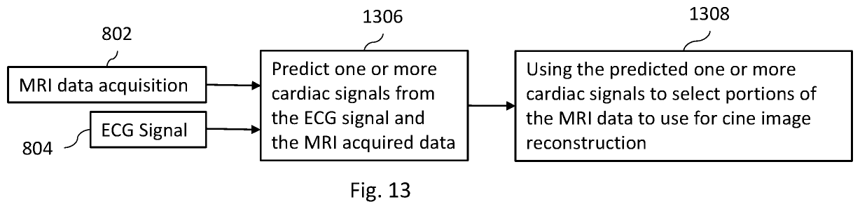

FIG. 7 illustrates an exemplary architecture of the processing engine 306 according to the disclosed embodiments. The processing engine 306 may include computer readable program code stored on at least one computer readable medium 702 for carrying out and executing the process steps described herein. The computer readable program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby, and Groovy, or other programming languages. The computer readable program code may execute entirely on the processing engine 306, partly on the processing engine 306, as a stand-alone software package, partly on the processing engine 306 and partly on a remote computer or server or entirely on the remote computer or server. In the latter scenario, the remote computer may be connected to the processing engine 306 through any type of network, including those mentioned above with respect to network 418.

The computer readable medium 702 may be a memory of the processing engine 306. In alternate aspects, the computer readable program code may be stored in a memory external to, or remote from, the processing engine 306. The memory may include magnetic media, semiconductor media, optical media, or any media which is readable and executable by a computer. The processing engine 306 may also include a computer processor 704 for executing the computer readable program code stored on the at least one computer readable medium 702. In at least one aspect, the processing engine 306 may include one or more input or output devices, generally referred to as a user interface 706 which may operate to allow input to the processing engine 306 or to provide output from the processing engine 306, respectively. The processing engine 306 may be implemented in hardware, software or a combination of hardware and software.

FIGS. 8-13 illustrate exemplary process flows according to aspects of the disclosed embodiments. In the process flows of FIGS. 8-12 MRI data may be acquired from any suitable MRI data source 802, for example, one or more of MRI data sources 502, 504, 506, 508, 510, and optionally ECG data 804 as will be explained below with respect to FIG. 13. The acquired MRI data is provided to the algorithm 304 which operates to predict cardiac cycles from the acquired MRI data 806. Sections of the acquired MRI data corresponding to selected portions of the predicted cardiac may then be operated upon using the processing engine. In some embodiments, the acquired MRI data may be reduced before being provided to the algorithm. For example, certain extraneous or unneeded cardiac phases or cardiac cycles may be removed that may reduce the data size to be processed by the algorithm and decrease computational time when using the algorithm and computational time of the processing engine 306.

In the exemplary process flow of FIG. 8, the operations include using the selected portions of the predicted cardiac cycle to position MRI data lines in the k-space 808. During cardiac MRI, the heart is beating and moving while scanning, making it difficult to capture a complete set of data points of the heart at any given time point. In practice, a complete cardiac cycle may be divided into several phases (for example, 20 phases in one cardiac cycle). For each phase (for example, the 10th phase), the MRI scanner 402 may only acquire a subset of the regions of the k-space because of difficulties in acquiring all the data at once. For example, in certain instances, only ⅓ of the whole k-space data may be acquired at a given time point. Using the cardiac cycle information, in the next cardiac cycle and the same phase (that is, the 10th phase in the 2nd cardiac cycle), the MRI scanner 402 may acquire another subset of the regions of the k-space (for example, another ⅓). Similarly, in the 10th phase in the 3rd cardiac cycle, the remainder of the k-space data may be acquired. Because the data generally repeats across the cardiac cycles, by using the predicted cardiac cycle and phase information the initial and subsequently acquired k-space lines may be placed into the proper corresponding positions.

In the exemplary process flow of FIG. 9, the acquired MRI data includes under sampled MRI data, and the operations include using the selected portions of the predicted cardiac cycle to interpolate between MRI data lines in the k-space 908. In a cardiac MRI scan, the user generally defines a number of cardiac phases into which each cardiac cycle may be divided, for example, the user can specify dividing each cardiac cycle into 25 phases. However, since each subject has a different time interval per cardiac cycle and the MRI scanner typically acquires k-space data at a set consistent rate different from a subjects cardiac cycle, the MRI scanner may only generate k-space data for a subset of the phases. In the example where each cardiac cycle is divided into 25 phases, the MRI scanner may only generate k-space data for 20 phases. As a result, data is interpolated between the phases to produce the specified number of phases, such as interpolating 20 phases into 25 phases. In order to perform the interpolation, the predicted cardiac cycle information for each phase and a predicted time interval between each phase is required.

In the exemplary process flow of FIG. 10, the acquired MRI data includes under sampled MRI data, and the operations include using the selected portions of the predicted cardiac cycle to interpolate between MRI images 1008. As mentioned above, the user generally defines a number of cardiac phases into which each cardiac cycle may be divided, for example, the user can specify dividing each cardiac cycle into 25 phases. However, since each subject has a different time interval per cardiac cycle and the MRI scanner typically generates image data at a set consistent rate different from a subjects cardiac cycle, the MRI scanner may only generate image data for a subset of the phases. In the example where each cardiac cycle is divided into 25 phases, the MRI scanner may only generate image data for 20 phases. As a result, data may be interpolated between the phases to produce the specified number of phases, such as interpolating 20 phases into 25 phases. In order to perform the interpolation, the predicted cardiac cycle information for each phase and a predicted time interval between each phase is required.

In the exemplary process flow of FIG. 11, the operations include performing strain analysis on the sections of the acquired MRI data 1108. In some embodiments, regions of interest may be tagged by creating locally induced magnetization perturbations using radiofrequency saturation panes. When the saturation pulses are applied in two orthogonal planes, the resulting tagging pattern forms a grid of intrinsic tissue markers, known as tags, that deform during contraction. Cardiac strain may be assessed by observing deformation of the tags.

As mentioned above, the MRI data acquired during MRI data acquisition 802 may be reduced before being provided to the algorithm 304 for predicting one or more cardiac signals from the MRI acquired data 806, thus reducing the data size to be processed by the algorithm 304. The reduction in acquired MRI data may also decrease the computational time of the processing engine 306 when performing cine image reconstruction 1208.

In the exemplary process flow of FIG. 12, the operations include performing cine image reconstruction on the sections of the acquired MRI data 1108. As mentioned above, the MRI data acquired during MRI data acquisition 802 may be reduced before being provided to the algorithm 304 for predicting one or more cardiac signals from the MRI acquired data 806, thus reducing the data size to be processed by the algorithm 304. The reduction in acquired MRI data may also decrease the computational time of the processing engine 306 when performing cine image reconstruction 1208.

Exemplary MR image reconstruction techniques may include using the predicted cardiac cycle and phase information to perform image reconstruction using one or more of a 2-dimensional Fourier transform technique, a back projection technique (e.g., a convolution back projection technique, a filtered back projection technique), an iteration reconstruction technique, etc. Exemplary iteration reconstruction techniques may include an algebraic reconstruction technique (ART), a simultaneous iterative reconstruction technique (SIRT), a simultaneous algebraic reconstruction technique (SART), an adaptive statistical iterative reconstruction (ASIR) technique, a model-based iterative reconstruction (MBIR) technique, a sinogram affirmed iterative reconstruction (SAFIR) technique, or the like, or any combination thereof.

Turning to the exemplary process flow of FIG. 13, MRI data may be acquired from any suitable MRI data source, for example, one or more of MRI data sources 502, 504, 506, 508, and 510. In addition, an ECG signal from a subject, corresponding to the acquired MRI data may also be collected. The acquired MRI data and the collected ECG data are provided to the deep learning model 308 which operates to predict cardiac cycles from the acquired MRI data. Sections of the acquired MRI data corresponding to selected portions of the predicted cardiac cycle portions are operated upon. In the exemplary process flow of FIG. 13, the operations include performing cine image reconstruction on the sections of the acquired MRI data 1308. In some embodiments, the MRI data acquired MRI data acquisition 802 may be reduced before being provided to the algorithm 304 for predicting one or more cardiac signals from the ECG signal and the MRI acquired data 1306, thus reducing the data size to be processed by the algorithm 304. The reduction in acquired MRI data may also decrease the computational time of the processing engine 306 when performing cine image reconstruction 1308 on the selected portions of the MRI data.

The process flow of FIG. 13, and the process flows of FIGS. 8-12 when utilizing the optional ECG signals, are advantageous in situations where cardiac function is compromised and the cardiac ECG signals are adversely influenced by the corresponding imperfect cardiac movement, structure, blood flow, or other cardiac characteristics. For these situations, the deep learning model may use the adversely influenced ECG signal and the acquired MRI data to more accurately predict the cardiac cycles. While the ECG signal may be adversely influenced, it still may provide useful information to the deep learning model for more accurately predicting the cardiac cycles and phases. It should be understood that while in the workflows of FIGS. 8-12, use of the ECG signals are optional, the MRI scanner 402 collects the ECG signals as a matter of operation, and that as a result, the ECG signals are available for use, whether utilized or not.

Thus, while there have been shown, described and pointed out, fundamental novel features of the invention as applied to the exemplary embodiments thereof, it will be understood that various omissions, substitutions and changes in the form and details of devices and methods illustrated, and in their operation, may be made by those skilled in the art without departing from the spirit and scope of the presently disclosed invention. Further, it is expressly intended that all combinations of those elements, which perform substantially the same function in substantially the same way to achieve the same results, are within the scope of the invention. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A method comprising:
acquiring MRI data;
using an algorithm to predict cardiac cycles from the acquired MRI data; and
operating on sections of the acquired MRI data corresponding to selected portions of the predicted cardiac cycles.

2. The method of claim 1, wherein the acquired MRI data includes one or more of k space data, image data, or under sampled MRI data.

3. The method of claim 1, wherein the acquired MRI data includes one or more of ECG signals, video images, or pulse data from a subject under study captured during MRI scanning.

4. The method of claim 1, wherein the algorithm comprises a deep learning model further comprising one or more of a combination CNN and RNN models, a GRU model, an LSTM model, a fully convolutional neural network model, a generative adversarial network, a back propagation neural network model, a radial basis function neural network model, a deep belief nets neural network model, an Elman neural network model.

5. The method of claim 1, wherein operating on sections of the acquired MRI data corresponding to selected portions of the predicted cardiac cycle comprises positioning data lines in a k-space of the acquired MRI data.

6. The method of claim 1, wherein operating on sections of the acquired MRI data corresponding to selected portions of the predicted cardiac cycle comprises interpolating between MRI data lines in a k-space of the acquired MRI data.

7. The method of claim 1, wherein operating on sections of the acquired MRI data corresponding to selected portions of the predicted cardiac cycle comprises interpolating between MRI images of the acquired MRI data.

8. The method of claim 1, wherein operating on sections of the acquired MRI data corresponding to selected portions of the predicted cardiac cycle comprises performing cardiac strain analysis using the sections of the acquired MRI data.

9. The method of claim 1, wherein operating on sections of the acquired MRI data corresponding to selected portions of the predicted cardiac cycle comprises performing cine image reconstruction on the sections of the acquired MRI data.

10. The method of claim 1, further comprising:
acquiring a cardiac signal corresponding to the MRI data; and
using the algorithm to predict the one or more predicted cardiac signals from the MRI acquired data and the acquired cardiac signal,
wherein operating on sections of the acquired MRI data corresponding to selected portions of the predicted cardiac cycle comprises performing cine image reconstruction on the sections of the acquired MRI data.

11. The method of claim 1, wherein the predicted portions of cardiac cycles represent any portions of the cardiac cycles.

12. The method of claim 1, wherein the predicted portions of cardiac cycles represent one or more of end systole cardiac phases, end diastole cardiac phases, P, Q, R, S, T, U, QRS complex, or PR interval cardiac phases.

13. A system comprising:
receiving and control circuitry operating an algorithm configured to predict cardiac cycles from MRI data; and
a processing engine configured to operate on sections of the MRI data corresponding to selected portions of the predicted cardiac cycles.

14. The system of claim 13, wherein the acquired MRI data includes one or more of k space data, image data, or under sampled MRI data.

15. The system of claim 13, wherein the acquired MRI data includes one or more of ECG signals, video images, or pulse data from a subject under study captured during MRI scanning.

16. The system of claim 13, wherein the algorithm comprises a deep learning model further comprising one or more of a combination CNN and RNN models, a GRU model, an LSTM model, a fully convolutional neural network model, a generative adversarial network, a back propagation neural network model, a radial basis function neural network model, a deep belief nets neural network model, an Elman neural network model.

17. The system of claim 13, wherein the processing engine operates on the sections of the acquired MRI data to position data lines in a k-space of the acquired MRI data.

18. The system of claim 13, wherein the processing engine operates on the sections of the acquired MRI data to interpolate between one or more of MRI images or MRI data lines in a k-space of the acquired MRI data.

19. The system of claim 13, wherein the processing engine operates on the sections of the acquired MRI data to perform cine image reconstruction on the sections of the acquired MRI data.

20. The system of claim 13, wherein the deep learning model is further configured to predict the cardiac cycles from a combination of the MRI data and a cardiac signal corresponding to the MRI data.
